(19) United States
(12) Patent Application Publication
(10) Pub. No.: US 2014/0248324 A1
(43) Pub. Date: Sep. 4, 2014

(54) NOVEL MULTIPHASIC BIOMATERIALS AND METHOD OF MANUFACTURING SAME

(71) Applicants: Laurent Jacques Grossin, Nancy (FR); Jean-Claude Voegel, Valff (FR); Pierre Gilbert Schaaf, Molsheim (FR); Pierre Gillet, Nancy (FR); Christel Odette Henrionnet, Toul (FR); Patrick Netter, Nancy (FR)

(72) Inventors: Laurent Jacques Grossin, Nancy (FR); Jean-Claude Voegel, Valff (FR); Pierre Gilbert Schaaf, Molsheim (FR); Pierre Gillet, Nancy (FR); Christel Odette Henrionnet, Toul (FR); Patrick Netter, Nancy (FR)

(73) Assignees: CENTRE NATIONAL DE LA RECHERCHE SCIENTIQUE, Paris Cedex 16 (FR); UNIVERSITE DE LORRAINE, Nancy (FR)

(21) Appl. No.: 14/115,009

(22) PCT Filed: Apr. 24, 2012
(86) PCT No.: PCT/FR2012/050895

§ 371 Date: Apr. 30, 2014

(30) Foreign Application Priority Data
Apr. 26, 2011 (FR) 1153563

(51) Int. Cl. A61L 27/52; A61L 27/38; A61L 27/20

(52) U.S. Cl. CPC A61L 27/52; A61L 27/20; A61L 27/38; A61L 2430/24
USPC 424/423; 623/23.72; 427/2.1; 424/93.7; 514/54; 424/602

(57) ABSTRACT

The novel biomaterial fillers containing cross-linked sodium alginate and the method for manufacturing same are used in the medical field and are intended for filling tissue lesions having a layered structure of varying compositions, such as cartilage, skin or the epithelium. These novel biomaterials have the special feature of being multiphasic, composite and functionalized, for medical use and, in particular, for treating tissue lesions having a layered structure of varying compositions. The biomaterials are particularly suitable for treating focal lesions of the articular cartilage.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention describes novel biomaterial fillers containing cross-linked sodium alginate for applications in the medical field and in particular intended for filling tissue lesions having a layered structure of varying compositions, such as cartilage, skin or the epithelium. These novel biomaterials have the special feature of being multiphasic, composite and functionalized, for medical use and in particular for treating tissue lesions having a layered structure of varying compositions. These biomaterials are particularly suitable for treating focal lesions of the articular cartilage. The method for manufacturing these novel biomaterials, implementing different steps of cross-linking the sodium alginate solution and a very particular method for depositing different layers, is described in detail. The different medical applications of these novel multiphasic, composite and functionalized biomaterials are also described.

2. Description of Related Art Including Information Disclosed Under 37 CFR 1.97 and 37 CFR 1.98.

One of the major challenges of the treatment of the lesions of the articular cartilage is essentially the creation of a substitute or filling multiphasic cartilaginous tissue. The treatment of the cartilaginous lesions is presently performed surgically according to several axes, among which the bone stimulation (for example micro-fracture surgery), mosaicplasty, periosteum or perichondrium grafts, osteochondral auto-grafts and allografts as well as cell-based repairs (for example transplantation of autologous chondrocytes). The bone stimulation techniques are intended for repairing the articular lesions through an arthroscopy. The lesion area to be filled and/or regenerated is perforated in order to expose the underlying bone. The sub-chondral bone is also perforated in order to generate a blood clot inside the injured portion containing mesenchymal stem cells, potential precursors of the bone and cartilage cells. One of the potential drawbacks of these methods is the insufficient filling of the chondral cavity. Furthermore, the tissue filler being obtained is often fibrocartilage having less good mechanical properties than the hyaline cartilage. In this case, the blood clot needs about 8 weeks to be transformed into fibrous tissue and needs 4 months to be transformed into fibrocartilage. This is not without any incidence on the rehabilitation and there exists an important risk of re-apparition of the symptoms 2 to 3 years after the initial operation. The fibrocartilage is indeed worn early because of the composition of its extracellular matrix, which is not made for withstanding the mechanical stresses applied during the stressing of the joint, which results into the necessity of a new surgical operation of the articular cartilage. Therefore, these bone stimulation techniques, and namely the micro-fracture surgery, are considered as intermediate, rather than final, therapies. Mosaicplasty implies the taking of small cylindrical bone sticks covered with healthy cartilage in a non-bearing area of the joint through an arthroscopy. Small perforations are then carried out at the spot of the cartilage lesion to be treated. The cylindrical sticks bearing healthy cartilage perfectly integrate into the so formed cavities. This technique is possible only with lesions not exceeding 2 to 3 cm², because it can only be contemplated to remove a very small quantity of healthy osteochondral tissue. This is the major drawback of this technique. The osteochondral autografts and allografts need transplanting sections of the bone and the cartilage. In a first step, the injured section of the bone and the cartilage is detached from the joint. Then a new healthy bone key with its cartilage is taken through perforation from the very joint and re-implanted into the cavity created by removing the old damaged bone with its cartilage. The healthy bone and its cartilage are taken from a non-bearing are of the joint, in order to avoid the dysfunction of the joint. Depending on the seriousness and the size of the lesion, different keys can eventually be required in order to repair the joint in a proper way. This process can prove difficult for an osteochondral auto-graft. In the case of an osteochondral allograft, the keys are taken from dead donors. This has the advantage of a larger quantity of osteochondral tissue being available and of lesions of a larger size being capable of being repaired. The drawback of this technique is the possibility of histocompatibility and ethical problems. The cell-based techniques (cell therapy, ACI for <<Autologous Chondrocyte Implantation>>) are based on the principle of replacing the damaged articular cartilage by autologous cartilage of the same type. The drawback of this technique is the generation of fibrocartilage or, in the best case, a combination of hyaline tissue and fibro-cartilaginous tissue, this being due to the absence of <<tutor materials>>. The autologous chondrocyte implantation processes are cell-based repair processes aimed at generating more functional hyaline neo-tissues. During the surgical intervention, chondrocyte cells are injected and applied on the injured area in combination with a membrane (periosteum). Each of these processes has advantages and drawbacks.

These techniques are heavy because they require a first operation for collecting the cells in a non-bearing area of the joint, then a second one for re-implanting into the injured area. These techniques generally provide good results. However, thanks to the development of the bio-engineering techniques, the use of biomaterials represents a serious alternative for the above-mentioned techniques (MACI for <<Matrix-induced Autologous Chondrocyte Implantation>>). The bio-engineering techniques, or tissue engineering, permit cell culture in artificial three-dimensional matrices having determined mechanical and biological properties close to those of the cartilaginous tissue. The artificial matrices described so far are of a synthetic, protein or polysaccharide nature. These techniques namely permit to obtain materials exhibiting a good mechanical strength, controllable biocompatibility and biodegradability, which are used as material fillers for treating in particular the focal lesions of the cartilage.

The patent application published under number WO 2010/116321 (Ginebra Molins et al.) describes a method for obtaining a composite calcium phosphate foam comprising the steps of: forming a polymeric foam by stirring or blowing gas in an aqueous polymer solution comprising gelatin, sodium alginate, a polymer from soya or combinations thereof; and mixing the foam obtained above with a calcium-phosphate cement powder. The invention also relates to the composite calcium phosphate foam that can be obtained by the method of the present invention and its use as biomaterial in the bone regeneration and/or as a scaffold for bone tissue engineering. This patent application describes a filling foam, which is composite and functionalized, but not multiphasic. Although a three-dimensional is formed, it is neither uniform nor homogenous and its thickness is difficult to be controlled.

The patent application published under number WO2010079496 describes a membrane comprising sodium alginate, at least one hydrophilic polymer and at least one plasticizer. This membrane is flexible and permits cell adherence, cell proliferation or cell differentiation. The invention relates in addition to the use of an inventive membrane for preparing implantable devices, among which cell-administration systems, cell-growth surfaces and biomaterials. The invention relates in addition to methods permitting to favor the tissue regeneration in an area including tissue substance losses, by applying the inventive membranes. These membranes can contain stem cells. The biomaterials according to the invention permit to fill lesions and/or cavities in ligament, tendon, cartilage, dental or bone tissues, but their three-dimensional structure is non-uniform and non-homogenous.

In order to meet the conditions necessary for obtaining an efficient biomaterial filler, it is necessary for said material, in addition to being multiphasic, to be porous in order to permit a cell colonization and to thus perform the functions it is intended for. Now, in order to provide a sodium alginate solution with a determined porosity, it is known from the prior art that its crosslinking should be proceeded to. This crosslinking is commonly carried out by means of calcium ions and namely of calcium chloride.

Sodium alginate, whether enriched or not, is at present commonly used for bioprinting or molding techniques (Guillemot et al., Biofabrication 2, 2010, 010201). Bioprinting can be defined as being the use of computer-aided transfer methods for creating and assembling living and non-living elements according to a given two-dimensional or three-dimensional structure, in order to create biocompatible structures likely to serve in the fields of regenerative medicine, pharmacokinetic studies or the basic cell biology studies. There are numerous publications implementing sodium alginate or calcium alginate based solutions, which are functionalized.

It has recently been shown that calcium sulfate (CaSO₄) is a perfectly suitable compound for cross-linking a sodium alginate solution. Sodium alginate is indeed a polysaccharide extracted from dried brown algae (Laminara Macrocystis). The monomers are D-mannuronic and L-glucuronic acids. It is commonly used as food additive under the name E401 as a texturing, emulsifying or gelling agent. In the presence of calcium ions, the sodium ions are shifted and take part in the forming of a network through polymerization. The various so formed polysaccharide chains for a gel. Specifically, this is a ionoreversible and non-thermoreversible regular three-dimensional geometric structure. The polymeric chains therein are parallel to each other. In practice, the texture and the quality of the gel depend on the ion concentration of the reaction medium, on the sodium alginate concentration and on its nature (namely its viscosity). All these mechanical and physical properties make it being a good candidate for developing a biomaterial intended for filling osteo-articular focal lesions. The use of three-dimensional matrices for creating a neo-cartilage in vitro using biomaterials for filling a loss of osteochondral substance is known (Frenkel S R et al., Front Biosciences, 1999, 4:671-685) from the prior art.

From the literature is namely known a method permitting to make an alginate gel (Tritz et al, Soft Matter, 2010), in which chondrocytes are dispersed. The cell suspension including the chondrocytes and the alginate solution is injected into a mold. The cross-linking is achieve by quickly dipping the mold including the suspension into a calcium chloride (CaCl₂) bath at 102 mM. This document also describes a pulverization system that can substitute the mold. This system is comprised of a gun, operating with a compressor, and it is connected to a container in which the solution alginate is located. Once it has been pulverized on a sterile glass plate, the cross-linked is, here too, achieved by dipping into a CaCl₂ bath at 102 mM.

This technique has however drawbacks: it is namely not possible to obtain a multiphasic biomaterial including a plurality of layers that can have a different composition, which are not mixed up, but interact between them. Indeed, according to this process, one single layer, with a small thickness, can be deposited, because of the extreme fluidity of the sprayed alginate solution. However, the material used in this document, and in particular the gun, would not permit to deposit a material having a higher viscosity; indeed, the nozzles of these guns have a diameter that is too small to permit a satisfactory spraying of a more viscous solution. In addition, if the viscosity of the solution were increased, the pressure of pulverizing by the gun would have to be increased, which would result into a prejudicial effect on the cells.

From the literature (Lee et al, Bioznaterials, 2007) is also known another method for manufacturing an alginate gel including chondrocytes. Prior to the molding step, the alginate solution is mixed with a highly concentrated calcium sulfate CaSO₄ solution (20 mg/mL), then deposited in a mold. It is possible to deposit two layers of alginate and, in this case, one of the layers is treated by means of a strip of paper impregnated with a citrate solution in order to favor an interaction of the alginate chains of the first layer with the second layer.

This technique has many drawbacks: on the one hand, it requires using citrate in order to favor an interaction between both layers of alginate; thus, it is not possible to obtain a continuity between the two layers. Because of the treatment with citrate, the method is heavy to be implemented and difficultly permits to obtain a biomaterial including a number of layers higher than two. Another drawback of this technique resides in that the diffusion of citrate by means of a pre-impregnated paper strip is not well-controlled. In addition, the citrate can have prejudicial effects on the cells dispersed in the alginate solution. Moreover, the high CaSO₄ concentration results into a complete cross-linking of the alginate, which makes its implementation difficult. Therefore, the method described by Lee et al. includes two cross-linking systems; the first one through addition of highly concentrated CaSO₄ and the second one through dipping the so obtained alginate gel is not optimal for its use as biomaterial filler. In addition, the very high CaSO₄ concentration is also likely to cause the forming of crystals that can have a prejudicial osteo-inducing effect on the cells encapsulated in the alginate solution.

From the prior art is also known a method developed by Grossin et al (Grossin et al., Advanced Material, 2009), in which the interaction between the layers of alginate is ensured by layers of polyelectrolytes, such as poly-L-lysine or hyaluronic acid, said layers alternating with layers of alginate incorporating the cells. This method is however heavy to be implemented and does not permit to obtain a continuity within the structure, because of the need to alternate several layers of alginate with layers of polyelectrolytes.

### SUMMARY OF THE INVENTION

The present invention describes novel biomaterial fillers based on cross-linked sodium alginate, permitting to cope with the drawbacks of the state of the technique, for applications in the medical field and in particular intended for filling tissue lesions having a layered structure of varying compositions, such as cartilage, skin or epithelium. The novel biomaterials according to the invention are multiphasic, composite and functionalized. This invention falls within the development of novel therapies based on the use of biomaterials containing autologous cells. The novel biomaterials according to the invention are easily adaptable to the dimensions of the lesion to be filled and their structure is close to that of the target tissue. This permits to contemplate a perfect integration into the target area and especially the generation of a filling tissue having good mechanical and biological properties. Furthermore, the novel biomaterial according to the invention also permits, when it is enriched, to contemplate the delivery of therapeutic molecules in a localized way, in order to reinforce the therapeutic arsenal existing so far (for example, anti-inflammatories). This cannot be contemplated with already structured prior-art material fillers, such as the collagen foams or sponges. Finally, according to the novel inventive method, the cells are homogenously distributed in the biomaterial. This is very original and can be contemplated only with difficulty with already structured materials, such as the collagen foams or sponges described in the prior art.

### BRIEF DESCRIPTION OF THE DRAWINGS

FIG. 1 is a schematic view, illustrating the method of manufacturing a cross-linked sodium alginate based biomaterial filler, according to embodiments of the present invention.

FIG. 2 is a graph illustration, showing viability of cells encapsulated in biomaterials by measuring mitochondrial activity.

### DETAILED DESCRIPTION OF THE DRAWINGS

The novel biomaterial fillers according to the invention are formed of a cross-linked sodium-alginate based solution deposited by means of airbrush pens. These various layers are deposited in an accurate and homogenous way, forming a multiphasic, composite and functionalized material, on an inert or functionalized support. This support is chosen depending on the target area to be treated. The present invention relates to a cross-linked sodium-alginate based biomaterial filler formed of at least two layers of cross-linked sodium-alginate deposited above each other thanks to airbrush pens that are preferably associated to a compressor.

The structure and the mechanical properties of the novel biomaterial depend on the nature of the sodium-alginate based solution, the consecutive crosslinking methods and the means for spraying the various layers of sodium alginate. The researchers have put into application a series of successive steps, including an initiation of the crosslinking by means of the calcium sulfate, then a crosslinking by means of the calcium chloride, in order to obtain a biomaterial filler having suitable mechanical properties. The invention describes a cross-linked sodium-alginate based biomaterial filler formed of at least two layers of sodium alginate, the crosslinking of which has been initiated by means of the calcium sulfate, deposited above each other thanks to airbrush pens and a compressor. The biomaterial filler according to the present invention preferably includes a number of layers higher than two. The so formed multiphasic biomaterial is subjected to a new crosslinking by means of the calcium chloride initiated through atomization by means of an airbrush pen and ended by a dip into a calcium chloride solution. This can be summarized by saying that the addition of calcium sulfate initiates the crosslinking of the sodium alginate into a hydrogel. The hydrogel being obtained permits to deposit several consecutive layers onto one and the same support. These layers will not be confused with each other, but will interact with each other, while remaining integral over time. Thus, the thickness of the final biomaterial can be much larger than what can be obtained with a simple sodium alginate hydrogel solution, i.e. without the crosslinking of the sodium alginate solution being initiated, as described in the literature (Tritz et si., Grossin et si.). According to the invention, the crosslinking of the gel is then carried out by an atomization step by means of calcium chloride and ended by a dip of the complete biomaterial, support included, into calcium chloride. The so obtained structure has a continuity that cannot be obtained when using additional solutions for treating the deposited layers in order to permit an interaction between the latter, namely by means of a citrate solution (Lee et al.) or polyelectrolytes (Grossin et al.).

By airbrush is understood an equipment capable of vaporizing a more or less viscous solution onto any surface by means of compressed air. An airbrush is thus combined with a compressor. In contrast to a “gun” that projects solutions, the airbrush permits to control both the air flow rate and the flow rate of the solution to be sprayed. In addition, the airbrush pen has a nozzle, the opening diameter of which is substantially larger than that of a gun nozzle, thus permitting a spraying of solutions having a determined viscosity, which is not the case with the traditionally used guns. In addition, an airbrush permits to carry out very accurate sprayings. The airbrushes are commonly used in the fields of painting and illustration, but also in the culinary field. The pens are the means for orienting the solution to be sprayed to the support being chosen. The pens are provided with nozzles, the diameter of which is chosen depending in the solution to be sprayed. In the present invention, Paasche® double action VL202-Set pens or Harder & Steenbeck® Colani Series pens and a compressor 30 1/mn, maximum 6 bars, are used.

By “functionalized biomaterial” is understood in the present invention the fact of making a biomaterial, which is generally inert and biocompatible, more suitable for adhesion and proliferation of the cells or the synthesis of an extracellular matrix by other elements being used. In the case of sodium alginate, the crosslinking creates a uniform network that permits the cell adhesion and development. This is a first level of functionalization. Surface treatments by means of various chemical compounds are also possible in order to optimize the functionalization of a sodium alginate based hydrogel. This constitutes a second level of functionalization. Finally, a third level of functionalization can be based on the association of a cell quota with the basic biomaterial, which will provide said biomaterial with new properties; said cells will indeed change the initial composition of the basic matrix by synthesizing their own extracellular matrix. The cell density and the cell phenotype can be adapted depending on the area to be regenerated (in order to regenerate an area of cartilage for example, it is useful to reproduce the zone structure of the hyaline articular cartilage).

By “functionalized support” is understood in the present invention the fact of making an inert solid support more suitable for adhesion and proliferation of the cells or other elements being used. The support will typically undergo a chemical modification through bioactive polymers or other compounds. The structure and/or the surface of the solid support can be functionalized.

By “composite biomaterial” is understood in the present invention the fact of depositing layers of biomaterials of different composition, the basis being sodium alginate, which can be associated with other molecules. As a non-restrictive example we will cite the addition of hydroxyapatite molecules, which, in addition to their biological activity, change the biochemical composition of the basic hydrogel.

By “multiphasic biomaterial” is understood in the present invention the fact of being able to construct structures by alternating consecutive layers the composition of which, both at the level of the associated materials (alginate, hyaluronic acid, chondroitin sulfates, hydroxyapatite) and at the level of the phenotypic characteristics of the cells and their density, can be modulated in order to better reproduce the layered structure of the hyaline articular cartilage. The biomaterial according to the invention includes at least two layers, but can include a number of layers higher than two, which can have different compositions, this number of layers depending namely on the size of the biomaterial one wants to obtain for permitting the filling.

The biomaterial according to the invention can be formed of a sodium alginate solution enriched with eukaryotic cells, but also with molecules having a biological activity, such as hyaluronic acid, the chondroitin sulfates or hydroxyapatite micro-particles (5-30 microns). The eukaryotic cells withstand the various crosslinking steps applied to the biomaterial during its elaboration. They remain viable after the steps of initiation of the crosslinking with calcium sulfate and perfectly adapt to the so formed three-dimensional network. The researchers carried out des viability studies in order to demonstrate that, in the case of articular chondrocytes, the latter do not exhibit any diminution of mitochondrial activity compared to eukaryotic cells encapsulated in a sodium alginate hydrogel prepared according to a traditional prior-art method (FIG. 2). The inventors have furthermore verified that the viability of the eukaryotic cells deposited in each layer of cross-linked sodium alginate is not affected by the successive depositions. Similar experiences have been carried out with mesenchymal stem cells proceeding from the bone marrow and have shown the same behavior before and after the crosslinking, i.e. no prejudicial effects of the process of preparation of the structure.

In the case of an enrichment with eukaryotic cells, the pressure exerted on the sodium alginate solution in the airbrush pens should be adapted to the survival of said eukaryotic cells. The exerted pressure should then vary between 1.0 and 1.5 bars. In a preferred embodiment, the pressure is 1.2 bars. In the case of an enrichment with eukaryotic cells, in order not to have a too high viscosity, the sodium alginate solution according to the invention is concentrated at 1.5% to 3.0%, preferably at 1.5% to 2.0%. In a particular embodiment, the biomaterial according to the invention is formed of a sodium alginate solution at 1.7%. The initiation of the crosslinking of this biomaterial is then carried out with a calcium sulfate (CaSO₄) solution. In order to permit such an initiation of the crosslinking, the CaSO₄ solution has advantageously a concentration between 1.0 and 10.0 mg/ml, more advantageously between 2.0 and 4.0 mg/ml and, in a particularly interesting way, this CaSO₄ concentration is substantially equal to 3.0 mg/ml.

In a particular embodiment, the cross-linking of the biomaterial is initiated with a calcium sulfate (CaSO₄) solution at a concentration of 3.0 mg/ml at the rate of 1.0 ml for 5.5 ml sodium alginate.

In the case of an enrichment with other elements, such as hyaluronic acid, chondroitin sulfates or hydroxyapatite particles, the viscosity of the solution increases with respect to the one described above. Therefore, either the sodium alginate concentration in the start solution should be reduced, or the diameter of the nozzle of the airbrush pens being used should be increased. Additional experiences have been carried out with sodium alginate solutions enriched with hyaluronic acid. The results have shown that a deposition can be carried out using a nozzle with a 1.2 mm diameter (Harder & Steenbeck® Colani series pen) while maintaining a pressure of 1.2 bars.

The present invention is based on an original method, which implements several techniques for cross-linking a sodium alginate solution. The successive implementation of these various cross-linking steps permits to obtain a composite, multiphasic and functionalized biomaterial filler that may be enriched or not. The present invention describes a novel method for manufacturing a cross-linked sodium alginate based biomaterial, which comprises the following steps:

a) preparing a sodium alginate based solution on the basis of 1.5% to 3.0%;

b) initiating the crosslinking of the sodium alginate based solution by adding a defined quantity in concentration and in volume of calcium sulfate;

c) depositing the so obtained solution on an inert or functionalized support by means of airbrush pens;

d) reiterating steps a) to c) in order to superimpose various layers of cross-linked sodium alginate on one and the same support;

e) once the desired thickness has been obtained, proceeding to initiate the final crosslinking of the biomaterial through atomizing calcium chloride by means of an airbrush pen;

f) proceeding to the final crosslinking by dipping the whole so obtained biomaterial into a bath of calcium chloride.

The airbrush pens and the compressor used for implementing the method according to the invention are traditional equipment known to the specialist in the art (Harder & Steenbeck® or Paasche® pens). The opening diameter of the pen is a determining element for the implementation of the method according to the invention, because, if the diameter is not large enough, the atomization of the sodium alginate solution, the cross-linking of which has now been initiated by adding calcium sulfate, is made difficult and results into a non-homogenous dispersion. An obstruction of the nozzles can indeed occur due to the viscosity of the sodium alginate based solution formed during step b) above. In general, the sodium alginate concentration determines the diameter of the nozzle of the pen to be used. In a particular embodiment, the nozzle of the airbrush pen should have a diameter between 1.0 mm and 1.2 mm. In a preferred embodiment, the airbrush pen has an opening diameter of 1 mm. If the biomaterial concentration being used is higher than 2.5%, a nozzle with a diameter larger than 1.2 mm should be used. Such pens seem however not to be available on the market today. Such an application is however contemplated in the present description.

The steps implementing the airbrush pens are performed under pressure. The compressor being used exerts a pressure between 1.0 and 1.5 bars. In a preferred embodiment, the compressor exerts a pressure of 1.2 bars.

Furthermore, the sodium alginate solution may be enriched with eukaryotic cells and/or with other molecules such as hyaluronic acid, chondroitin sulfates or hydroxyapatite particles.

In the case of an enrichment with eukaryotic cells, the pressure exerted in the airbrush pens must preserve these cells and not be too high, in order not to affect their survival and development. The sodium alginate solution should then be concentrated at 1.5% to 2.0%. In a particular embodiment, the method for manufacturing a cross-linked sodium alginate based biomaterial filler according to the invention implements a sodium alginate based solution enriched with eukaryotic cells and concentrated at 1.7% with sodium alginate. As described above, the eukaryotic cells withstand the various cross-linking steps exerted on the biomaterial being elaborated. They remain viable after the steps of initiating the cross-linking with calcium sulfate and perfectly adapt to the so formed three-dimensional network. The researchers carried out viability studies in order to demonstrate that, in the case of chondrocytes, the latter do not exhibit any diminution of mitochondrial activity, compared to eukaryotic cells encapsulated in a sodium alginate hydrogel prepared according to a traditional prior-art method (FIG. 2). The inventors have furthermore verified that the viability of the eukaryotic cells deposited in each layer of cross-linked sodium alginate is not affected by the successive depositions.

If the sodium alginate solution is enriched with other molecules, such as hyaluronic acid, the chondroitin sulfates or the hydroxyapatite particles, the sodium alginate concentration should be reduced. These other molecules indeed increase the viscosity of the solution obtained in step b) of the method according to the invention. The technique of deposition by means of airbrush pens can be used only with a determined viscosity range. It can also be contemplated to use pen nozzles with a larger diameter. However, some technical limits exist and it is difficult to find nozzles having a diameter larger than 1.2 mm. Such an equipment can however be developed in order to adapt the method according to the invention to solutions having a higher viscosity.

The support used in step c) of the method according to the invention for depositing the sodium alginate based solution of step b) of the method according to the invention can be inert or functionalized. In a particular embodiment, the support is functionalized in order to obtain a biomaterial filler having optimal mechanical and biological properties.

The researchers have surprisingly discovered that a very interesting biomaterial filler is obtained by implementing the method according to the invention comprising the following steps:

a) preparing a sodium alginate based solution on the basis of 1.5% to 3.0%, said solution being enriched with eukaryotic cells;

b) initiating the crosslinking of the sodium alginate based solution by adding a calcium sulfate solution having a concentration between 1.0 and 10.0 mg/ml;

c) depositing the so obtained solution on an inert or functionalized support by means of airbrush pens under a pressure between 1 and 1.5 bars;

d) reiterating steps a) to c) in order to superimpose various layers of cross-linked sodium alginate on one and the same support;

e) once the desired thickness has been obtained, proceeding to initiate the final crosslinking of the biomaterial through atomizing calcium chloride by means of an airbrush pen, the calcium chloride concentration being between 80 and 120 mM;

f) proceeding to the final crosslinking by dipping the whole so obtained biomaterial into a bath of calcium chloride, the concentration of which is between 80 and 120 mM.

According to a preferred exemplary embodiment, the concentration of the calcium sulfate solution used in step b) is between 2.0 mg/ml and 4.0 mg/ml, and is yet more preferably substantially equal to 3.0 mg/ml.

Preferably, the calcium sulfate is added on the basis of 1 ml for 3 to 8 ml sodium alginate solution, preferably of 1 ml for 5 to 6 ml. More advantageously, the volume used for crosslinking the sodium alginate solution is 1.0 ml for 5.5 ml sodium alginate.

According to an interesting embodiment, the pressure implemented in step c) of the method is substantially equal to 1.2 bars.

Preferably, the calcium chloride concentration of steps e) and f) is substantially equal to 102 mM.

Thus, in a particularly advantageous way, the method according to the invention comprises the following steps:

a) preparing a sodium alginate based solution at 1.7%, enriched with eukaryotic cells;

b) initiating the crosslinking of the sodium alginate based solution by adding calcium sulfate at 3 mg/ml on the basis of 1 ml for 5.5 ml sodium alginate solution;

c) depositing the so obtained solution on an inert or functionalized support by means of airbrush pens under a pressure of 1.2 bars;

d) reiterating steps a) to c) in order to superimpose various layers of cross-linked sodium alginate on one and the same support;

e) once the desired thickness has been obtained, proceeding to initiate the final crosslinking of the biomaterial through atomizing calcium chloride at 102 mM by means of an airbrush pen;

f) proceeding to the final crosslinking by dipping the whole so obtained biomaterial into a bath of calcium chloride at 102 mM.

This original method combines several factors that are determining for the quality of the biomaterial according to the invention: the homogenous deposition by means of the airbrush pens in a sodium alginate based solution enriched with eukaryotic cells, a pulverization pressure suitable for the eukaryotic cells and the crosslinking in several steps, including namely an initiation of the crosslinking, of the various layers being deposited. The so obtained biomaterial has all the properties necessary for filling focal lesions of the human or animal cartilage. One of the major difficulties of the method for depositing through atomization of a sodium alginate based hydrogel resides in that the air pressure used for depositing the viscous biomaterial solution results into causing the solution to flow outside the support. This difficulty is avoided by using calcium sulfate to initiate the crosslinking of the solution to be deposited, the direct consequence of which is the obtaining of a deposition surface that is less sensitive to this air pressure. This thus permits to deposit more substance and, hence, to obtain a larger thickness. This is completely impossible when using a sodium alginate solution the crosslinking of which has not been initiated prior to the deposition.

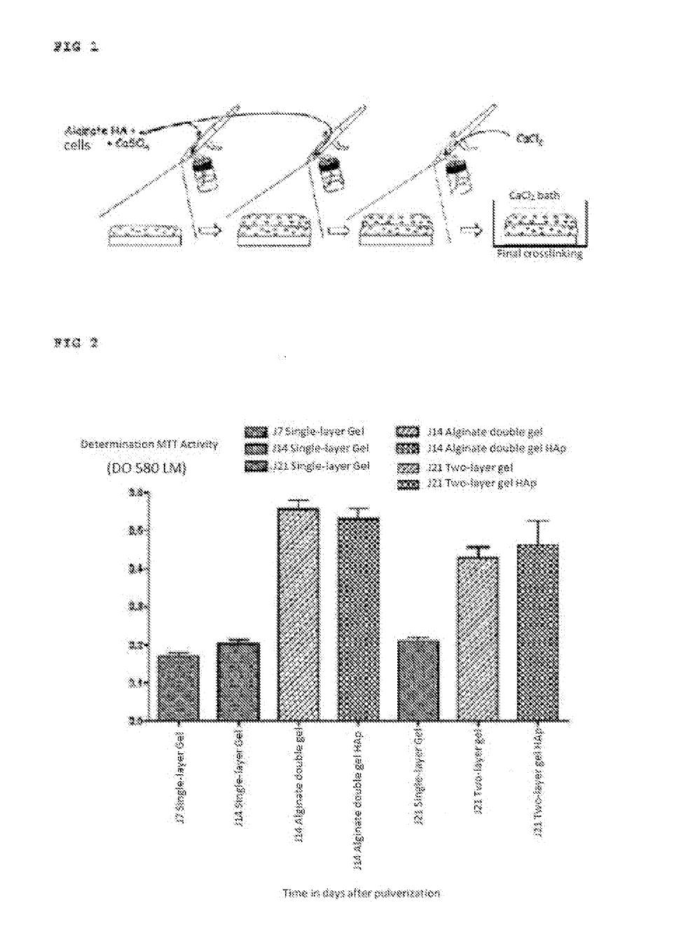

The present invention is shown by the following figures. FIG. 1, which describes the steps of the method for manufacturing a cross-linked sodium alginate based biomaterial filler enriched with eukaryotic cells:

(1) Deposition of the first layer of a sodium alginate solution, calcium sulfate and eukaryotic cells by means of airbrush pens;

(2) Deposition of the second of a sodium alginate solution, calcium sulfate and eukaryotic cells by means of airbrush pens;

(3) Initiation of the crosslinking through atomization of a CaCl₂ solution by means of airbrush pens;

(4) Final crosslinking of the biomaterial by dripping into a CaCl₂ solution.

FIG. 2 shows the viability of the cells encapsulated in (single-layer and two-layer) biomaterials according to the prior art by measuring the mitochondrial activity.

The implementation of any equivalent means for carrying out the method according to the invention should be considered as falling within the scope of the present patent application.

The present invention is implemented and described in detail in the examples below and the corresponding figures. These examples are in no way restrictive as regards the scope of the invention described and claimed herein.

### Examples

### Manufacture of a Non-Enriched Biomaterial

All the equipment is sterilized by dipping into a 70% ethanol solution. The alginate solution is sterilized by autoclaving at 121° C. for 20 minutes. It is then recovered with a 0.9% NaCl solution. The concentration of the so obtained sodium alginate is 2%, i.e. 2 g for 100 ml. The sodium alginate solution is stirred for 24 hours.

An extemporaneous CaSO₄ solution is prepared at a concentration of 3 mg/ml and filtered on a 0.22 μm membrane. In an atomization cup, previously sterilized and rinsed with a sterile NaCl solution, a volume of 5.5 ml of sodium alginate is mixed with 1 ml of a CaSO₄ solution, then actively mixed, in order to obtain a solution the viscosity of which is compatible with the atomization.

A first deposition is carried out with an aerograph spray at a pressure of 1.2 bars at a distance of 10.0 cm to 15.0 cm from the inert support laying down. The use of a functionalized support can be contemplated. Once the complete volume of sodium alginate solution combined with the CaSO₄ is deposited in one uniform layer and after waiting for 5 minutes, the second layer is deposited according to the same modalities as before.

Once the desired number of successive layers has been reached, the whole material so created is subjected to an atomization with a CaCl₂ solution for 20 seconds. A new crosslinking is then initiated. This permits a manipulation of the biomaterial during its elaboration.

The biomaterial is then placed in a CaCl₂ bath at a concentration of 102 mM for a period of time between 20 and 25 minutes, depending on the number of layers of cross-linked sodium alginate being deposited (about 10 minutes per deposited layer).

The various steps of this method are shown in detail in FIG. 1.

2. Manufacture of a Biomaterial Enriched with Eukaryotic Cells

The whole equipment is sterilized by dipping in a 70% ethanol solution. The alginate solution is sterilized by autoclaving at 121° C. for 20 minutes. It is then recovered with a 0.9° A) NaCl solution. The concentration of the so obtained sodium alginate is 2%, i.e. 2 g for 100 ml. The sodium alginate solution is stirred for 24 hours.

An extemporaneous CaSO₄ solution is prepared at a concentration of 3 mg/ml and filtered on a 0.22 μm membrane.

In order to functionalize or enrich the biomaterial, the selected cells are recovered by trypsination and washed. They are then associated with the sodium alginate solution at a concentration of 3.106 cells/ml alginate. A sodium alginate solution enriched with eukaryotic cells is obtained.

In an atomization cup, previously sterilized and rinsed with a sterile NaCl solution, a volume of 5.5 ml of sodium alginate is enriched with eukaryotic cells, mixed with 1 ml of a CaSO₄ solution, then actively mixed, in order to obtain a solution the viscosity of which is compatible with the atomization.

A first deposition is carried out with an aerograph spray at a pressure of 1.2 bars at a distance of 10.0 cm to 15.0 cm from the support laying down. This support may be inert or functionalized. Once the complete volume of sodium alginate solution enriched and cross-linked with the CaSO₄ is deposited in one uniform layer and after waiting for 5 minutes, the deposition of the second layer is performed according to the same modalities as before.

Once the desired number of successive layers has been reached, the whole material so created is subjected to an atomization with a CaCl₂ solution for 20 seconds. A new crosslinking is then initiated. This permits a manipulation of the biomaterial during its elaboration.

The biomaterial is then placed in a CaCl₂ bath at a concentration of 102 mM for a period of time between 20 and 25 minutes, depending on the number of layers of cross-linked sodium alginate being deposited (about 10 minutes per deposited layer). This bath should not exceed 30 minutes, because the eukaryotic cells would exhibit a largely reduced viability rate.

The various steps of this method, with the exception of the step of enrichment with eukaryotic cells, are shown in detail in FIG. 1.

3. Analysis of the Viability of the Cells Encapsulated in (Single-Layer and Two-Layer) Biomaterials by Measuring the Mitochondrial Activity (MTT Test).

This test is based on the mitochondrial activity of the viable cells: the MTT (3-[4,5-dimethylthiazol-2-yl]-2,5-diphenyl-tetrazolium bromide), of yellow color, is reduced in the presence of the mitochondrial dehydrogenase succinate in the form of blue formazan crystals. The cells being used here are human chondrocytes in primo-culture.

A biopsy calibrated with gel containing sodium alginate, human chondrocytes and enriched or not with hydroxyapatite (HAp), is placed in a well, with 125 ?l complete medium and 25 ?l MTT solution (Sigma®) at 5 mg/ml diluted in PBS. The plates are incubated for 4 hours at 37° C. The liquid is eliminated, then replaced by a SDS-DMF (Sodium Dodecyl Sulfate; DiMethylFormamide H₂O; pH 4.7) buffer permitting to lyse the balls. The whole is incubated for 24 hours at 37° C. The absorbance at 580 nm is measured by means of a spectrophotometer (Multiskan EX, ThermoLabsystems).

Single-layer or two-layer gels are prepared and the mitochondrial activity is measured during the days following the pulverization of the cells on the various gels.

The results of the MTT test are shown in FIG. 2. One can observe that the cells have a mitochondrial activity in a gel made of sodium alginate based biomaterial until 21 days after their pulverization. These cells are therefore viable.

1. Method for manufacturing a cross-linked sodium alginate based biomaterial filler, the method comprising the steps of:
a) preparing a sodium alginate based solution on a basis of 1.5% to 3.0%;
b) initiating crosslinking of the sodium alginate based solution by adding a defined quantity in concentration and in volume of calcium sulfate;
c) depositing an obtained solution on an inert or functionalized support by airbrush pens;
d) reiterating steps a) to c) in order to superimpose various layers of cross-linked sodium alginate on the support;
e) once desired thickness has been obtained, proceeding to initiate final crosslinking of biomaterial through atomizing calcium chloride by an airbrush pen;
f) proceeding to final crosslinking by dipping a whole obtained biomaterial into a bath of calcium chloride.

2. Method for manufacturing a cross-linked sodium alginate based biomaterial filler according to claim 1, wherein the calcium sulfate solution has a concentration between 1.0 and 10.0 mg/ml, preferably between 2.0 and 4.0 mg/ml, preferably substantially equal to 3.0 mg/ml.

3. Method for manufacturing a cross-linked sodium alginate based biomaterial filler according to claim 1, wherein the calcium sulfate is added on the basis of 1 ml for 3 to 8 ml sodium alginate solution, preferably of 1 ml for 5 to 6 ml, the calcium sulfate is preferably added on the basis of 1 ml for 5.5 ml sodium alginate solution.

4. Method for manufacturing a cross-linked sodium alginate based biomaterial filler according to claim 1, wherein initiation of the final crosslinking is obtained through atomizing calcium chloride having a concentration between 80 and 120 mM, preferably substantially equal to 102 mM.

5. Method for manufacturing a cross-linked sodium alginate based biomaterial filler according to claim 1, wherein the final crosslinking is obtained by dipping into a calcium chloride having a concentration between 80 and 120 mM, preferably substantially equal to 102 mM.

6. Method for manufacturing a cross-linked sodium alginate based biomaterial filler according to claim 1, wherein the support is functionalized.

7. Method for manufacturing a cross-linked sodium alginate based biomaterial filler according to claim 1, wherein the sodium alginate based solution is enriched with eukaryotic cells and concentrated at 1.7% sodium alginate.

8. Method for manufacturing a cross-linked sodium alginate based biomaterial filler according to claim 1, wherein a minimum opening diameter of the nozzle of the aerograph pen is between 1.0 mm and 1.2 mm, preferably 1.0 mm.

9. Method for manufacturing a cross-linked sodium alginate based biomaterial filler according to claim 1, further comprising the following steps:
a) preparing a sodium alginate based solution at 1.7%, enriched with eukaryotic cells;
b) initiating crosslinking of the sodium alginate based solution by adding calcium sulfate at 3 mg/ml on the basis of 1 ml for 5.5 ml sodium alginate solution;
c) depositing an obtained solution on an inert or functionalized support by airbrush pens under a pressure of 1.2 bars;
d) reiterating steps a) to c) in order to superimpose various layers of v on one and the same support;
e) once desired thickness has been obtained, proceeding to initiate the final crosslinking of the biomaterial through atomizing calcium chloride at 102 mM by an airbrush pen;
f) proceeding to the final crosslinking by dipping a whole obtained biomaterial into a bath of calcium chloride at 102 mM.

10. Cross-linked sodium alginate based biomaterial filler obtained according to claim 1, said filler being multiphasic and comprising at least two layers of sodium alginate crosslinking of which has been initiated by calcium sulfate, said layers being deposited above each other by airbrush pens.

11. Cross-linked sodium alginate based biomaterial filler according to claim 10, wherein said at least two layers of sodium alginate are deposited above each other by airbrush pens and a compressor, wherein formed multiphasic biomaterial is subjected to a new crosslinking by the calcium chloride initiated through atomization by an airbrush pen and ended by a bath in a calcium chloride solution.

12. Biomaterial according to claim 10, wherein the sodium alginate solution is enriched with an element selected from a group consisting of: eukaryotic cells, molecules having a biological activity, hyaluronic acid, chondroitin sulfates, and hydroxyapatite micro-particles.

13. Biomaterial according to claim 10, wherein the sodium alginate concentration is between 1.5% and 3.0%, preferably 1.7%.

14. Biomaterial according to claim 10, wherein the calcium sulfate solution has a concentration between 1.0 and 10.0 mg/ml, preferably between 2.0 and 4.0 mg/ml, preferably substantially equal to 3.0 mg/ml.

15. Biomaterial according to claim 14, wherein volume used for crosslinking of the sodium alginate solution is 1 ml for 3 to 8 ml sodium alginate solution, preferably 1 ml for 5 to 6 ml, preferably 1 ml for 5.5 ml sodium alginate.
